(19) United States
(12) Patent Application Publication
(10) Pub. No.: US 2013/0267883 A1
(43) Pub. Date: Oct. 10, 2013

(54) METHOD, APPARATUS, AND SYSTEM FOR TREATMENT OF BLOOD OF A PATIENT

(71) Applicant: Guillermo MEDRANO, Schweinfurt (DE)

(72) Inventor: Guillermo MEDRANO, Schweinfurt (DE)

(73) Assignee: Fresenius Medical Care Deutschland GmbH, Bad Homburg (DE)

(21) Appl. No.: 13/618,778

(22) Filed: Sep. 14, 2012

Related U.S. Application Data
(60) Provisional application No. 61/538,991, filed on Sep. 26, 2011.

(30) Foreign Application Priority Data
Sep. 26, 2011 (DE) 10 2011 053 935.2

(51) Int. Cl. A61M 1/14; A61M 1/34; A61M 1/28

(52) U.S. Cl. CPC A61M 1/14; A61M 1/28; A61M 1/34
USPC 604/5.01; 604/28; 604/500; 604/503; 604/246; 604/66; 604/29

(57) ABSTRACT

The present invention relates to a method for treating the blood of a patient, comprising removing water from the blood of patients at a settable water removal rate, wherein the position of the patient is determined and the water removal rate is set, controlled, and/or regulated depending on the determined position of the patient. The invention further relates to an apparatus for treating the blood of a patient, comprising a blood treatment device for removing water from the blood of the patient at a settable water removal rate, wherein position-determining means for determining the position of the patient, wherein the water removal rate is settable, regulatable, and/or controllable depending on the determined position of the patient.

### TECHNICAL FIELD

The invention relates to a method for treating blood of a patient and to an apparatus and a system for treating blood.

### PRIOR ART

Various methods for treating blood of a patient, in particular for removing water from the blood of a patient, are known.

For example, in hemodialysis, the blood of a patient is cleaned in an extracorporeal blood circuit, which includes a dialyzer. The dialyzer comprises a blood chamber through which the blood of the patient is passed and a chamber for dialysis fluid. The blood chamber and the chamber for the dialysis fluid are separated by means of a semipermeable membrane. During hemodialysis, the dialysis fluid flows through the dialysis fluid chamber and the blood of the patient flows through the blood chamber. Due to a concentration gradient between the dialysis fluid and the blood of the patient, corresponding substances are transported through the semipermeable membrane due to diffusion.

Peritoneal dialysis is a blood treatment method which is not conducted extracorporeal but within the patient. In peritoneal dialysis, the peritoneal cavity of the patient is filled with a dialysis fluid via a catheter guided through the abdominal wall, wherein the dialysis fluid has an appropriate concentration with regard to the body's own fluids.

Toxins located in the body transfer into the dialysis fluid located in the abdominal cavity through the peritoneum which then acts as a membrane. After some time, typically several hours, the henceforth used-up dialysis fluid located in the abdominal cavity of the patient is exchanged.

Removing water from the blood of the patient also occurs via the concentration gradient of soluble substances between blood and dialysis fluid. For this purpose, the dialysis fluid is enriched, for example, with glucose, dextrins, and/or other substances. The concentration of these substances may be settable, for example, over time, for concentrates by means of mixing devices (pumps, valves, mixing chambers), so that targeted removal of water may be performed. For at least partial automation of this method, particular machines are employed in practice, such as, e.g., the “Sleep Safe” unit of Fresenius Medical Care.

Blood treatment, for example, with one of the above described methods, is inter alia necessary when kidney failure occurs in a patient. In this case, dialysis must be performed on the patient so that waste products, such as urea, creatinine, and uremic toxins, may be removed from the blood of the patient. Furthermore, during dialysis, excess water and other substances, which are usually obligatorily excreted by urine, are removed from the body of the patient.

A commonly employed dialysis format and method is extracorporeal hemodialysis, in which the blood of the patient flows along a dialysis membrane, wherein a dialysis fluid is provided on the other side of this semi-permeable dialysis membrane, wherein the dialysis fluid flows countercurrent to the blood stream in the majority of the methods.

Through this semi-permeable membrane, the substances which are to be removed from the blood of the patient are removed due to a concentration gradient between the blood and the dialysis fluid, wherein these substances diffuse through the semipermeable membrane. Thereby, larger molecules which have a very low diffusion velocity may also be transported convectively through the semipermeable membrane by means of fluid flow from the blood side to the side of the dialysis fluid.

Thereby, the dialysis solution for extracorporeal hemodialysis is typically provided so that such a concentration of certain substances to be removed from the blood is provided that the appropriate concentration gradient from the blood side towards the side of the dialysis fluid is provided.

In another known method, namely hemofiltration, certain substances are filtered out due to a pressure gradient across a membrane of a dialysis filter. For this method as well, the porosity and/or permeability of the semipermeable membrane is accordingly set so that, at the given pressure gradient, the corresponding substances are filtered from the blood and are removed via the membrane. However, with regard to the respective molecular sizes, it is clear that, in hemofiltration, in addition to the partially large molecular substances, which are supposed to diffuse through the semipermeable membrane due to the pressure gradient, such substances are also removed from the blood and/or filtered out that are physiologically essential for the blood composition. For example, electrolytes are removed from the blood in hemofiltration. Accordingly, in hemofiltration, the excessively removed substances and the excessively removed fluid volume are reintroduced to the blood of the patient after it has passed through the dialysis filter by means of a substitution infusion fluid.

A combined approach also exists, which is known as hemodiafiltration, wherein in this method the above mentioned stages of hemodialysis and hemofiltration are inserted into the extracorporeal circuit either simultaneously or sequentially.

In the dialysis methods, in addition to removal of substances usually obligatorily excreted by urine, excess volume of water is removed from the blood and/or the body of the patient. The removal of water from the blood of the patient due to a pressure difference across the dialysis membrane is also referred to as ultra-filtration. This pressure difference, which is used to generate the ultra-filtration, is often generated by an ultra-filtration pump arranged downstream of the respective dialysis filter on the side of the dialysis fluid.

Furthermore, it is known that during execution of the blood treatment a patient should preferably be at rest to avoid jeopardizing the success of the blood treatment and, in particular, not to excessively burden the cardiovascular system of the patient. In particular due to the removal of a large volume, i.e., a large removal of water from the blood of the patient, a large cardiovascular stress may occur, which culminates, in an extreme case, in a hypotension emergency. For prevention thereof, the patient is preferably kept at rest.

Maintaining the position of a patient during blood treatment, for example, by means of a patient bed, which is provided with a blood treatment device, is known in accordance with DE 101 41 053 A1. The therein described patient bed is designed to provide the patient with a reasonably comfortable seated/lying position during the blood treatment, which usually lasts several hours.

### DETAILED DESCRIPTION OF THE INVENTION

Accordingly, the objective forming the basis of the present invention is to provide a method for treating the blood of a patient and an apparatus and a system for performing the blood treatment, in which occurrence of cardiovascular-related complications is further reduced.

The objective is solved by the method according to claim 1. Advantageous embodiments of the method result from the dependent claims.

Accordingly, the method for treating the blood of a patient comprises removing water from the blood of the patient at a settable water removal rate. According to the invention, the position of the patient is determined and the water removal rate is set, controlled, and/or regulated in dependence of the determined position of the patient.

Herein, the water removal rate is to be understood as the fluid volume removed from the blood of the patient per unit of time, i.e., in particular the water removed from the blood of the patient volume per unit of time. Depending on the applied blood treatment method (e.g., hemodialysis, hemofiltration, hemodiafiltration, peritoneal dialysis, apheresis, and/or plasmapheresis), the water removal rate substantially equals the ultra-filtration rate if removing water from the blood is achieved by ultra-filtration.

It has turned out that setting, controlling, and/or regulating the fluid removal and/or the water removal rate as accurately as possible is extremely important for dialysis in order to prevent and/or control complications or liquid removal problems arising in the patient, such as cardiovascular problems, as far as possible. Then, at the same time, a relatively fast dialysis process may be achieved by setting, controlling, and/or regulating the water removal rate if an optimal water removal rate may be used for the respective patient state.

Due to the removal of water, a decrease in plasma volume occurs. If the respective human organism of the patient fails to replenish the plasma volume from the interstitial space, the filling pressure of the heart correspondingly declines and the blood pressure drops. It is immediately understood that the higher the correspondingly set water removal rate is the more this effect applies. The blood volume is thereby regarded as the total blood volume, which is composed of the plasma volume and the erythrocytes volume. In case the blood pressure drops too much, a hypotension emergency occurs in the respective patient undergoing treatment.

It has been shown that the body position of the patient influences the plasma volume. In particular, the plasma volume increases in a lying position and contrastingly decreases in a sitting and/or standing position. Accordingly, the case may arise that a patient, who is treated at a constant water removal rate and who moves from a lying position into a more upright position during the blood treatment, experiences a sudden drop in blood pressure. Consequently, the body position of the patient directly influences the plasma volume.

The above described solution to the present objective, namely, in a method for treating the blood of a patient, in which water is removed from the blood of the patient at a settable water removal rate, measuring the position of the patient and setting, regulating, and/or controlling the water removal rate in dependence of the position of the patient has an effect to the extent that the water removal rate will accordingly be reduced when the patient, for example, rises or sits up from his bed.

Correspondingly, the occurrence of cardiovascular-related problems, which are due to the change in the position of the patient, may be reduced or avoided in this way. By means of the described method for treating blood, therefore, the occurrence of critical hypotension states in the patient and, in particular, the occurrence of a hypotension emergency in the patient may be reduced and/or avoided.

The determination of the position of the patient is preferably performed on a patient bed, on a patient chair, and/or on the patient itself. Preferably, it is determined by evaluation of the sensors mounted to the patient bed and/or to the patient chair, of sensors arranged on the patient and/or of sensors monitoring the patient, in particular by evaluation of position sensors, optical sensors, and/or imaging sensors.

Preferably, removing water from the blood is performed by means of a peritoneal dialysis, and the water removal rate is set, controlled, and/or regulated by setting, controlling, and/or regulating the concentration of dissolved substances in the dialysis fluid, preferably of glucose and/or dextrin, before introducing the dialysis fluid into the abdominal cavity. In order to achieve further adaption of the water removal rate to the current state of the patient and, in particular, to the positional state of the patient, the dialysis fluid located in the abdominal cavity is preferably exchanged for a dialysis fluid with a different concentration of dissolved substances when the position of the patient changes.

Preferably, removing water from the blood is performed by ultra-filtration, wherein the water removal rate is settable by means of the ultra-filtration rate (UFR), and wherein the ultra-filtration rate (UFR) is set, controlled, and/or regulated in dependence of the determined position of the patient.

The regulation of the removal of water based on the detected position of the body of the patient may result in shorter dialysis times, particularly, if the patient is primarily lying down in the course of the treatment. After all, in the lying position, a higher plasma volume is usually obtained in the patient; therefore, the water removal rate may be increased. A correspondingly shortened treatment protects the patient and, in the case of the peritoneal dialysis, his/her peritoneum acting as a filter.

The regulation of the removal of water based on the detected position of the body of the patient may also result in lower glucose concentrations in the dialysis fluid, if the body position results in a lower plasma volume. A reduced glucose concentration contributes to a lower glucose load on the peritoneum. It has been shown that, in different patients, an elevated glucose concentration in the dialysis fluid may result in unwanted pathological changes of the peritoneum over time, which may prevent peritoneal dialysis treatment in extreme cases. Therefore, a reduced glucose concentration due to the detected position of the body may contribute to the fact that a patient may be treated with the method of peritoneal dialysis for longer periods.

Setting, controlling, and/or regulating of the ultra-filtration rate may be advantageously achieved during ultra-filtration by setting, controlling, and/or regulating the pumping rate, in particular by setting, controlling, and/or regulating the pumping rate of a hose reel pump, gear pump, diaphragm pump, and/or impeller pump arranged downstream of a dialysis filter.

In a preferred variant, for a substantially lying patient, the water removal rate, preferably the ultra-filtration rate, is set, controlled, and/or regulated on a higher level than for a patient being arranged in a more upright position with regard to the lying position. In this way, the water removal rate may precisely be adapted to the physiological conditions so that low-complication and efficient removal of water from the blood of the patient may be achieved.

Preferably, the water removal rate is increased with regard to the previous water removal rate when the position of the patient approximates a more lying posture, and the water removal rate is decreased when the position of the patient changes more towards an upright position with regard to the previous position.

For this purpose, a position measurement is preferably determined on a patient bed, a patient chair, and/or on the patient himself/herself by means of corresponding sensors and/or imaging means.

In another preferred embodiment, the patient may himself/herself adjust his body position, in particular by means of actuators arranged at a corresponding patient bed or patient chair used by him/her. This ensures that, during blood treatment, the relatively long treatment time may be arranged as comfortable as possible for the patient. In particular, the patient is capable of continually and independently adjusting his/her body position. For adjustment of the body position, which is controlled by the patient himself/herself, the water removal rate, preferably the ultra-filtration rate, is simultaneously adjusted so that, even for a changed body position, no problems arise, such as a hypotension emergency of the patient. By means of the patient's extensive self-control over his/her own position, treatment for each individual patient may accordingly be arranged more comfortable.

The method is preferably employed for hemodialysis, hemofiltration, hemodiafiltration, peritoneal dialysis, apheresis, and plasmapheresis.

In order to achieve fast treatment of the blood of the patient with little complications, the water removal rate, preferably the ultra-filtration rate, is preferably set, controlled, and/or regulated at a higher level at the beginning of treatment than at a subsequent phase of the treatment. Thereby, the physiological conditions may also be taken into account to the extent that the excess plasma volume is greatest at the beginning of the treatment.

In another advantageous embodiment of the method, the water removal rate, preferably the ultra-filtration rate, is controlled and/or regulated in dependence of the position of the patient only in a first treatment phase but is kept constant in a second treatment phase, wherein the first treatment phase preferably ends by removing half of the planned amount of liquid. Accordingly, the second phase of the treatment substantially resembles conventional treatment.

In another advantageous embodiment of the method, the water removal rate, preferably the ultra-filtration rate, is raised and/or lowered from a predetermined water removal rate, preferably ultra-filtration rate (UFR_{SET}), by a predetermined difference (UFR+) for setting, regulating, and/or controlling it in dependence of the position of the patient, wherein the predetermined difference (UFR+) is preferably derived from the expected change in plasma volume between a substantially lying and substantially upright position of the patient. In this way, the occurrence of a postural hypotension emergency may especially well be avoided because the water removal rate is adapted to the expected plasma volume change. The described procedure accordingly leads to the result that the water removal rate is precisely adapted to the expected plasma volume change, and safe and efficient removal of water may accordingly be carried out.

In another embodiment of the process, wherein the water removal rate, preferably the ultra-filtration rate (UFR), is initially raised for a period (t_{UFR±}) of the treatment, preferably by a predetermined value (UFR+), and, in the event of a change in position of the patient towards an upright position, is subsequently reduced for the same period (t_{UFR±}), preferably by the same predetermined value (UFR+), wherein, initially, an increased water removal rate, preferably ultra-filtration rate (UFR=UFR_{SET}+UFR+), and, subsequently, a reduced water removal rate, preferably ultra-filtration rate (UFR=UFR_{SET}−UFR+), is preferably set, regulated, and/or controlled. By employing the initially increased and subsequently decreased water removal rate, a treatment may be provided, in which, compared to the conventional methods, treatment duration is not extended, but safety regarding potential hypotension emergencies is increased.

In another advantageous embodiment, the water removal rate is increased with regard to the conventionally used value at the beginning of each individual treatment and is adapted to the position of the patient only in the course of treatment. In particular, the water removal rate may only be adapted after exceeding a certain inclination angle of the patient, for example, the upper body of the patient forming an angle of greater than 30° with regard to the horizontal plane. Accordingly, in a range between a fully horizontal orientation of the body of patient and this angle, no adaptation is carried out.

Furthermore, the individual water removal rate may also be chosen by taking into account the typical behavior of the patient, thus by taking into account the time that the patient typically spends in a more upright position. Accordingly, in order to observe the overall treatment time, the water removal rate may be increased by an amount, which derives from the prediction of expected time spent in the more upright state, at the start of treatment and in sections, in which the patient is in a more upright position.

The above described objective is further achieved by a device with the features of claim 14. Advantageous embodiments are described in the dependent claims.

Correspondingly, the apparatus for treating the blood of a patient comprises a blood treatment apparatus for removing water from the blood of the patient at a settable water removal rate. According to the invention, position-determining means for determining the position of the patient are provided, wherein the water removal rate is settable, regulatable, and/or controllable in dependence of the determined position of the patient.

Preferably, the position-determining means are configured as sensors mounted to the patient bed and/or to the patient chair, as sensors arranged at the patient and/or as sensors monitoring the patient, and are preferably configured as position sensors, optical sensors, and/or imaging sensors.

For providing the patient with high treatment comfort, actuators acting on a patient bed and/or on a patient chair are provided, by means of which the patient may himself/herself adjust his/her position.

A data processing apparatus for evaluating the signals of the position-determining means may be provided, wherein the data processing apparatus may regulate and/or control the water removal rate, preferably the ultra-filtration rate (UFR), corresponding to the determined position of the patient.

The blood treatment apparatus is preferably configured for performing hemodialysis, hemofiltration, hemodiafiltration, peritoneal dialysis, apheresis, and/or plasmapheresis.

Furthermore, the above-described objective is achieved by a system with the features of claim 20.

### BRIEF DESCRIPTION OF THE FIGURES

In the following, preferred embodiments of the present invention are described in the following Figures:

FIG. 1 schematically shows a device for treating blood, having a patient bed and a blood treatment apparatus;

FIG. 2 shows the course of ultra-filtration in a conventional method according to the prior art;

FIG. 3 schematically shows the course of ultra-filtration in a method according to an embodiment of the present invention; and

FIG. 4 schematically shows an exemplary algorithm for performing the present method.

### DETAILED DESCRIPTION OF THE FIGURES

In the following, the present invention will be described in detail with reference to the Figures. Thereby, same reference numerals are used for similar or identical components, and, in the different embodiments, repeated description thereof can be omitted in order to avoid redundancies.

In the following, the method, the apparatus, and the system are exemplarily described using the example of hemodialysis. This description is not to be regarded as limiting; on the contrary, the method and the apparatus and the system may be used in any other blood treatment, in particular in hemodialysis, hemofiltration, hemodiafiltration, peritoneal dialysis, apheresis, and/or plasmapheresis.

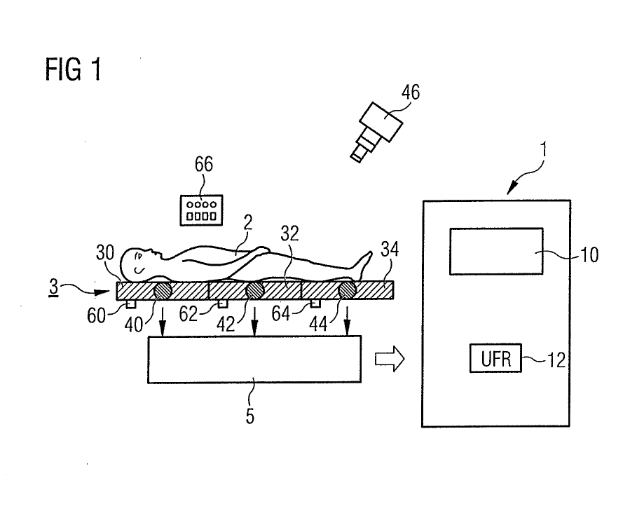

In FIG. 1, an apparatus for treating the blood of a patient according to a preferred embodiment of the present invention is schematically shown.

For this purpose, a blood treatment apparatus 1 is provided in the form of a hemodialysis machine, by means of which the patient 2 may be subjected to blood cleaning and/or hemodialysis. The hemodialysis machine comprises a water removal apparatus in the form of the ultra-filtration section 10, which may be operated at a settable water removal rate in the form of a settable ultra-filtration rate UFR.

Accordingly, by setting the ultra-filtration rate UFR of the blood treatment device 1, the fluid removal in patient 2 may be set during treatment. Thereby, the ultra-filtration rate UFR equals the volume of the fluid removed by means of ultra-filtration, herein referred to as UFV, per time t, i.e., equals UFR=UFV/t. In general, the water removal rate is the water volume removed from the blood of the patient per unit time.

The patient 2 is supported by a patient bed 3. In the shown embodiment, the patient bed 3 includes a head portion 30, a middle portion 32, and a foot portion 34. The head portion 30, the middle portion 32, and the foot portion 34 may be placed in different positions relative to each other so that the patient 2 may further be raised from the completely horizontally oriented—i.e., lying-position shown in FIG. 1. The patient 2, for example, may be raised by raising or tilting the head portion 30 so that his/her upper body is brought into a more upright position with regard to a lying position. The foot portion 34, for example, may simultaneously be lowered so that the individual components of the patient bed 3 then provide a supporting position for the patient 2, in which the patient 2 rather assumes a sitting position, i.e., with raised upper body and lowered legs. The foot portion 34 may also be raised in order to correspondingly bring back blood volume from the legs to the core area of the patient 2 in the event of cardiovascular problems.

The patient bed 3 may further be slightly tiltable as a whole, i.e., the entire patient 2 may be brought into a tilted position in order to correspondingly provide the patient 2 with a tilted bed.

The patient bed 3 shown in FIG. 1 is basically a conventional patient bed, which provides the corresponding adjustment possibilities. The individual parts of the patient bed 3 are thereby displaced relative to each other by means of actuators 60, 62, 64 in order to allow for a simple change of the position of patient 2. The actuators 60, 62, 64 are typically provided in the form of electric motors, pneumatic actuators, and/or hydraulic drives. In a preferred variant, the patient 2 may himself/herself actuate the actuators 60, 62, 64 by means of a corresponding control panel 66 in order to obtain a comfortable position for him/her.

Preferably, the patient bed 3, at least for using the patient bed 3 in an upright position or in a position similar to a sitting position, is provided with an arm-receiving component, which is not shown in FIG. 1, intended to provide the patient 2 with armrests.

In FIG. 1, the patient bed 3 is shown as consisting of only three sections, namely of the head portion 30, the middle portion 32, and the foot portion 34. However, the patient bed 3 may be configured to be of any complexity and, in particular, may comprise even more sections, which are adjustable with regard to each other, than those shown in FIG. 1, for example, in addition to a separate head portion, an adjustable back portion or a split footrest, to allow for more different positions of the patient 2, to provide him/her with the most comfortable support during the blood treatment. To this regard, it should be noted that the blood treatment usually takes a relatively long time—treatment durations of 3 to 7 hours are typical. Therefore, it is particularly important for the well-being of the patient 2 that he/she may obtain a comfortable position for this time.

Also, the blood treatment apparatus 1 in the form of a hemodialysis machine, which is shown in FIG. 1, is a largely conventional hemodialysis machine, in which the ultra-filtration rate UFR may be set by a corresponding setting unit 12.

The blood treatment apparatus 1 may also be configured for performing hemofiltration, hemodiafiltration, peritoneal dialysis, apheresis, and/or plasmapheresis.

At the patient bed 3, sensors 40, 42, 44 are provided, by means of which the position of each part of the treatment bed 3 may be determined with regard to the horizontal plane and/or with regard to each other. Furthermore, a camera 46 is provided, which is also used for determining the current position of the patient 2.

By means of evaluating the signals from the sensors 40, 42, 44, 46, the exact position of the patient 2 with regard to the horizontal plane may be determined in a data processing device 5. Naturally, prerequisite for deriving the posture and/or position of the patient 2 from the particular setting of the patient bed 3 is that the patient 2 assumes the position provided by the form of the individual parts of the patient bed 3. In other words, it is assumed that the patient 2 substantially aligns to the individual components of the patient bed 3 in the manner provided and/or rests on these components.

However, if the patient 2 rises independently, for example, in the positioning of the patient bed 3 shown in FIG. 1, and the upper body is consequently no longer in contact with the patient bed 3, the position of the patient 2 can naturally not be derived from the position of the patient bed 3. In the following, it is, however, assumed that the patient is always in contact with the respective components of the patient bed 3 during the blood treatment.

In a variant or addition, the position of the patient 2 may also be determined by means of sensors arranged directly on the patient, for example, sensors incorporated in the respective textiles worn by the patient 2.

In another preferred variant or addition, optical sensors and/or imaging sensors, such as the shown camera 46, may be used for determining the position of the patient 2. Thereto, for example, photoelectric measurements or laser scanner measurements, or image recordings and/or video recordings of the patient during the blood treatment, from which the particular situation of the patient 2 may be determined, may be suitable.

In the data processing apparatus 5, the position of the body of the respective patient 2 is accordingly determined by means of the sensors 40, 42, 44, 46, shown in FIG. 1, and/or by means of other possible sensors, not shown in FIG. 1.

The water removal rate is set in the dialysis machine 1 in dependence of the position of the patient 2, which is determined in the data processing device 5. Correspondingly, the ultra-filtration rate UFR is accordingly set in the hemodialysis apparatus 1 with ultra-filtration section 10, herein shown as an example.

Because it has been shown that the plasma volume of a patient 2 changes with regard to his/her position, it may be achieved in this way that the ultra-filtration rate UFR is always optimally adapted to the particular state of the patient 2. In this way, detrimental effects on stability of the cardiovascular system of the treated patient may be reduced and/or avoided.

In particular, it has been found that the plasma volume of a patient 2 increases when he/she is moved from a more upright position into a more lying position and the plasma volume decreases when the patient 2 is moved from a more lying position into a more upright position.

The water removal rate, in particular the ultra-filtration rate UFR, is correspondingly set, regulated, and/or controlled on the basis of the determined position and/or the respective change in position of the patient 2 by the data processing device 5.

The respective changes effected to the water removal rate and/or the ultra-filtration rate UFR in response to a change in the position of the patient 2 may be calculated, for example, from the expected change in plasma volume between a lying and an upright position of the patient 2. As an alternative, a relation between the change in position of the patient 2 and a statistically determined adaptation of the water removal rate is employed. This statistical analysis may either be performed for multiple patients or, however, for each individual patient. For determining this data, for example, other parameters are monitored in the first sessions of a patient, for example, the blood pressure of the patient. From correlating the individual parameters, in particular the position of the patient and the blood pressure, the appropriate adjustment factor may then be calculated.

Setting, controlling, and/or regulating the water removal rate on the basis of the determined position of the patient 2, which is performed by the data processing device 5, is achieved either via a fixed mathematical relation between the position of the patient 2 and the water removal rate or performed by reading from a table statistically generated for certain position ranges.

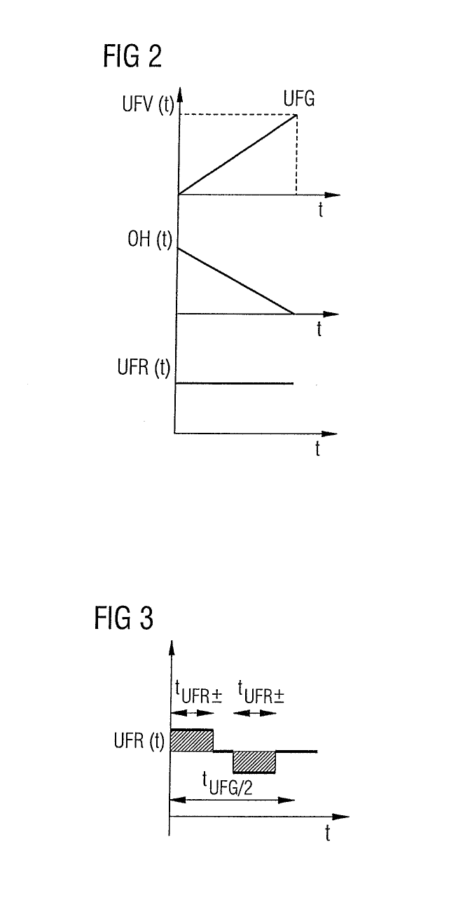

FIG. 2 schematically shows the course of a conventional dialysis treatment by means of a hemodialysis apparatus with an ultra-filtration section according to the prior art, wherein, in the top graph, the total ultra-filtration volume is plotted over time as UFV(t). Hereby, it is seen that the ultra-filtration volume, i.e., the volume of fluid taken from the patient, increases quasi linearly over time until the desired or planned ultra-filtration volume UFG is achieved.

In the middle diagram arranged below, the excess volume of water in the patient 2 over time is plotted over time as OH(t). At the end of each blood treatment, the excess volume of water OH in the patient should have dropped to zero, then, as reflected in the diagram arranged above, the desired ultra-filtration volume UFG has been obtained.

In the bottommost diagram, the ultra-filtration rate over time is indicated as UFR (t). It is directly seen that the ultra-filtration rate is kept constant in the conventional method.

FIG. 3 now shows a possible course of the water removal rate, in particular of the ultra-filtration rate, over time as UFR(t) according to a possible embodiment of the present invention. It is evident that the ultra-filtration rate UFR(t) in this shown embodiment is variable. In particular, at the beginning of treatment, the ultra-filtration rate UFR(t) is initially increased by an amount UFR+ over a period of time t_{UFR±}.

A substantial disturbance of the cardiovascular system of the patient 2 is not expected in this very first phase of the blood treatment because, here, the plasma volume is at its highest yet and, accordingly, a decrease in plasma volume associated with an increased water removal rate and/or ultra-filtration rate UFR does not result in a hypotension emergency. In addition, at the beginning of treatment, the patient 2 typically assumes a lying position.

The time t_{UFR±} is a statically determined time, over the course of which a patient typically holds a more upright position, for example, an inclination of the upper body of greater than 30° with regard to the horizontal plane. The value t_{UFR±} either is determined from statistics of a particular number of different patients or may be determined from the previous sessions of the respective individual patient 2. It should be noted that dialysis patients typically must attend a dialysis session two to three times a week, and a significant data base is accordingly quickly gathered for each individual patient.

Then, as is clear from FIG. 3, the water removal rate and/or ultra-filtration rate UFR is later reduced by the value UFR+ when the patient changes his/her position. Thereby, lowering of the water removal rate and/or ultra-filtration rate UFR is performed again over the course of the previously determined period t_{UFR±} after the patient has changed his/her position.

In the embodiment shown herein, in FIG. 3, an adaptation to the respective change in position may only be performed as long as half of the planned water removal volume and/or ultra-filtration volume UFG has not been obtained. In other words, the adaptation is performed only if the time corresponding to the desired half of the total amount of ultra-filtration UFG/2, which is herein referred to as t_{UFG/2}, has not been obtained. The time, at which half of the water removal volume and ultra-filtration volume is obtained, is referred to as t_{UFG/2}.

Then, after this period has elapsed, the originally specified water removal rate and/or ultra-filtration rate, which is hereinafter referred to as UFR_{SET}, is employed for the subsequent removing of water.

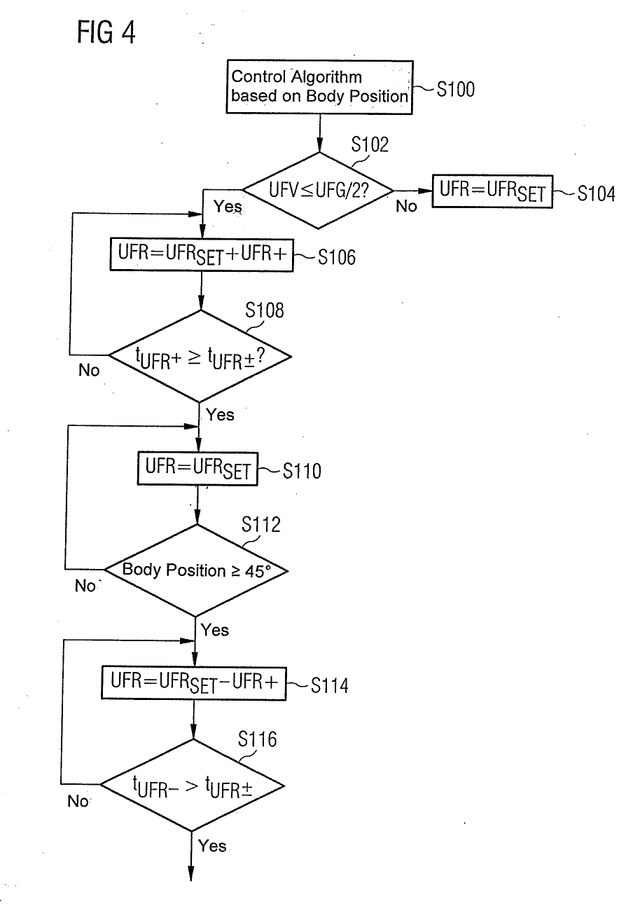

FIG. 4 shows a corresponding algorithm, by means of which the water removal rate is determined.

First, as step S100, the control algorithm based on body position is provided.

In an initial query, in step S102, it is determined whether the water removal volume and/or ultra-filtration volume UFV already obtained is still less than half of the planned water removal volume and/or ultra-filtration volume UFG, i.e., the query UFG≦UFV/2. If this condition is not met, i.e., the already obtained water removal volume and ultra-filtration volume UFV of patients is greater than half of the planned water removal volume and/or ultra-filtration volume UFG, the water removal rate and/or ultra-filtration rate is set to the originally specified water removal rate and/or ultra-filtration rate, i.e., UFR=UFR_{SET}, in step S104.

If the already obtained water removal volume and/or ultra-filtration volume UFV is still less than half of the planned water removal volume and/or ultra-filtration volume UFG, the water removal rate and/or ultra-filtration rate is set to an increased value in step S106, namely by a change value of the water removal rate or ultra-filtration rate, adapted to the change in body position, which is herein referred to as UFR+. Accordingly, the water removal rate and ultra-filtration rate in step S106 is then UFR=UFR_{SET}+UFR+.

In the subsequent query in step S108, it is checked whether the time, in which this increase in the water removal rate and/or ultra-filtration rate is performed, is still less than the expected time of the upright position of the patient. Accordingly, it is checked whether t_{UFR+}≧t_{UFR±}. If this time has not expired yet, the increase in the water removal rate and/or ultra-filtration rate by the value UFR+ is maintained. If this time has elapsed, the water removal rate and ultra-filtration rate is decreased to the predetermined water removal rate and ultra-filtration rate UFR_{SET}. Accordingly, UFR=UFR_{SET} is set in step S110.

In the next query, in step S112, it is checked whether the body position of the patient has changed in the meantime, in particular to a value of ≧45°, i.e., whether the patient has risen up.

If this is the case, the water removal rate and/or ultra-filtration rate is decreased in step S114, namely again by the amount UFR so that the water removal rate and/or ultra-filtration rate is set to UFR=UFR_{SET}−UFR+ in step S114.

In the next query at step S116, it is then checked whether the time for reducing the water removal rate and/or ultra-filtration rate, namely t_{UFR-1} already the predetermined time for the average retention period of patients in the upright position, namely t_{UFR±}, has already elapsed. If this is not the case, i.e., t_{UFR-}≧t_{UFR±}, the water removal rate and/or the ultra-filtration rate is maintained at the value lowered by the amount UFR+. However, once this period has elapsed, the water removal rate and/or ultra-filtration rate is again maintained at the determined water removal rate and/or ultra-filtration rate, i.e., UFR_{SET}.

The advantage of this algorithm is that a time loss and/or a prolongation of treatment does not occur because the phase of the reduced water removal rate and/or ultra-filtration rate (UFR=UFR_{SET}−UFR+) is compensated for by the initial phase with the increased water removal rate and/or ultra-filtration rate (UFR=UFR_{SET}+UFR+). Thereby, it is assumed that, at the beginning of treatment, the patient is lying and, consequently, a high volume of plasma is available so that circulatory system problems do not occur, even at the increased water removal rate and/or ultra-filtration rate. At a later stage, prior to half the treatment duration, the treatment may, however, be supported and improved in view of the occurrence of a hypotension emergency in that that the water removal rate and ultra-filtration rate is reduced when the patient rises up.

The algorithm shown in FIG. 4 works particularly well if the position of the patient is changed only once during the treatment from a lying position into an upright position. Further development of this algorithm with regard to constant monitoring and corresponding adaptation of the water removal rate to the respective position of the patient is, however, immediately feasible for the skilled person. In particular, especially if prolongation of the treatment is unproblematic, a direct correlation between the position angle of the patient with regard to the horizontal plane and the water removal rate may be established. Hereby, a higher water removal rate is possible when the patient is lying, i.e., at an angle of 0°, compared to when a patient raised up, for example, at a position of the backrest of greater than 30° and close to 90°. The corresponding relation may here be found by means of a statistical analysis across different patients or from historic-statistical data for each individual patient.

In a particularly preferred variant, the patient 2 may himself/herself determine, by means of the switch panel 66, the position, in which he/she wishes to perform the respective periods of his/her blood treatment. The patient may adjust, for example, via the switch panel 66, the respective positions of the patient bed 3 in a known manner so that he/she may assume the most comfortable position for him/her.

The position of the patient bed selected and/or set by the patient is then used by the data processing device 5 to set the water removal rate at the corresponding position. Here again, a different water removal rate is also set in dependence of the angle of the patient with regard to the horizontal plane, wherein the water removal rate is higher for a lying patient as the water removal rate for a patient seated upright. In this way, the treatment for the patient 2 may be designed significantly more pleasant because he can freely dispose over his/her position.

The treatment duration may slightly be extended due to the free choice in view of position. The water removal rate may, however, be increased with regard to the conventional water removal rate for a lying patient because the otherwise normally fixed water removal rate is predetermined for both the lying patient and for patient being in a more upright position. A patient being in a lying position may, however, withstand a higher water removal rate without resulting in a hypotension emergency. Consequently, even for such a setting and for a pre-selection of the respective position of the patient in direct coupling to the water removal rate, a treatment duration can be achieved, which is bearable for the patient and economically viable for the respective medical facility.

1. A method for treating blood of a patient (2), comprising removing water from the blood of the patient (2) at a settable water removal rate,
characterized in that
the position of the patient (2) is determined and the water removal rate is set, controlled, and/or regulated in dependence of the determined position of the patient (2).

2. The method according to claim 1, wherein determining the position of the patient (2) is performed on a patient bed (3), on a patient chair and/or on to the patient (2) himself/herself, preferably by the evaluation of sensors (40, 42, 44) mounted to the patient bed (3) and/or to the patient chair, of sensors arranged on the patient (2), and/or of sensors monitoring the patient (2), particularly by the evaluation of position sensors (40, 42, 44), optical sensors, and/or imaging sensors (46).

3. The method according to claim 1, wherein removing water from the blood is performed by means of a peritoneal dialysis and the water removal rate is set, controlled, and/or regulated by means of setting, controlling, and/or regulating the concentration of substances dissolved in the dialysis fluid, preferably of glucose and/or dextrin, prior to introducing the dialysis fluid into the abdominal cavity.

4. The method according to claim 3, wherein the dialysis fluid located in the abdominal cavity is replaced for a dialysis fluid, which has a different concentration of dissolved substances, when the position of the patient (2) changes.

5. The method according to claim 1, wherein removing water from the blood is performed by ultra-filtration, wherein the water removal rate is settable via the ultra-filtration rate (UFR), and the ultra-filtration rate (UFR) is set, controlled, and/or regulated in dependence of the determined position of the patient (2).

6. The method according to claim 5, wherein the setting, controlling, and/or regulation of the ultra-filtration rate (UFR) is achieved by means of setting, controlling, and/or regulating the pumping rate during the ultra-filtration, in particular by setting, controlling, and/or regulating the pumping rate of a hose reel pump, gear pump, diaphragm pump, and/or impeller arranged downstream of a dialysis filter.

7. The method according to claim 1, wherein the water removal rate, preferably the ultra-filtration rate, is set, controlled, and/or regulated at a higher level for a substantially lying patient (2) than for a patient (2) arranged in a position more upright with regard to the lying position.

8. The method according to claim 1, wherein the patient (2) adjusts his/her position himself/herself, especially by means of actuators acting on a patient bed (3) and/or a patient chair.

9. The method according to claim 1, wherein the water removal rate, preferably the ultra-filtration rate, is set, controlled, and/or regulated at a higher level at the start of treatment than at a subsequent section of the treatment.

10. The method according to claim 1, wherein the water removal rate, preferably the ultra-filtration rate, is controlled and/or regulated in dependence of the position of the patient (2) only in a first treatment phase; in a second treatment phase, however, it is kept constant, wherein the first treatment section preferably ends by withdrawing half of the planned amount of liquid (UFG/2).

11. The method according to claim 1, wherein the water removal rate, preferably the ultra-filtration rate, for setting, controlling, and/or regulating in dependence of the position of the patient (2) is raised and/or lowered from a predetermined water removal rate, preferably ultra-filtration rate (UFR_{SET}), by a predetermined difference (UFR+), wherein the predetermined difference (UFR+) is preferably derived from the expected change in plasma volume between a substantially lying and a substantially upright position of the patient (2).

12. The method according to claim 1, wherein the water removal rate, preferably the ultra-filtration rate (UFR), is initially raised for a period (t_{UFR±}) of the treatment, preferably by a predetermined value (UFR+), and, in the event of a change in position of the patient towards a more upright position, is then lowered for the same period (t_{UFR±}), preferably by the same predetermined value (UFR+), wherein, preferably initially an increased water removal rate, preferably ultra-filtration rate (UFR=UFR_{SET}+UFR+), and then a reduced water removal rate, preferably ultra-filtration rate (UFR=UFR_{SET}−UFR+), is set, regulated, and/or controlled.

13. The method according to claim 1, wherein the method is employed in hemodialysis, hemofiltration, hemodiafiltration, peritoneal dialysis, apheresis and/or plasmapheresis.

14. An apparatus for treating the blood of a patient (2), comprising a blood treatment apparatus (1) for removing water from the blood of the patient (2) at a settable water removal rate,
characterized by
position determining means (40, 42, 44, 46) for determining the position of the patient (2), wherein the water removal rate is settable, regulatable, and/or controllable in dependence of the determined position of the patient.

15. The apparatus according to claim 14, wherein the position determining means are configured as sensors (40, 42, 44) mounted to the patient bed (3) and/or to the patient chair, as sensors arranged at the patient (2) and/or as sensors monitoring the patient and preferably configured as position sensors, optical sensors, and/or imaging sensors (46).

16. The apparatus according to claim 14, wherein actuators acting on a patient bed (3) and/or on a patient chair are provided, by means of which the patient (2) is capable of independently setting his/her position.

17. The apparatus according to claim 14, wherein a data processing device (5) for evaluating the signals of position determining means (40, 42, 44, 46) is provided, wherein the data processing apparatus (5) is configured for setting, regulating, and/or controlling the water removal rate, preferably the ultra-filtration rate (UFR), corresponding to the determined position of the patient (2).

18. An apparatus for treating the blood of a patient (2), comprising a blood treatment apparatus (1) for removing water from the blood of the patient (2) at a settable water removal rate, characterized by position determining means (40, 42, 44, 46) for determining the position of the patient (2), wherein the water removal rate is settable, regulatable, and/or controllable in dependence of the determined position of the patient, wherein control means for performing a method according to claim 1 is provided.

19. The apparatus according to claim 14, wherein the blood treatment apparatus (1) is configured for performing hemodialysis, hemofiltration, hemodiafiltration, peritoneal dialysis, apheresis and/or plasmapheresis.

20. A system for treating blood, comprising a blood treatment apparatus (1) for removing water from the blood of a patient (2) at a settable water removal rate, and a patient bed (3) and/or a patient chair for supporting the patient (2) during the blood treatment,
characterized in that
position-determining means (40, 42, 44) for determining the position of the patient (2) are provided, and a data processing device (5) for evaluating the signals of the position-determining means (40, 42, 44) is provided, wherein the data processing device (5) is configured for setting, regulating, and/or controlling the water removal rate according to the determined position of the patient (2).
